(19) United States
(12) Patent Application Publication
(10) Pub. No.: US 2012/0214177 A1
(43) Pub. Date: Aug. 23, 2012

(54) METHODS AND COMPOSITIONS FOR DIAGNOSIS OF UROSEPSIS AND URINARY TRACT INFECTION

(75) Inventors: Jonathan Barasch, New York, NY (US); Catherine Forster, Wayne, PA (US); Thomas L. Nickolas, New York, NY (US); Neal Paragas, New York, NY (US)

(73) Assignee: The Trustees of Columbia University in the City of New York, New York, NY (US)

(21) Appl. No.: 13/378,986

(22) PCT Filed: Jun. 17, 2010
(86) PCT No.: PCT/US10/39018

§ 371 Date: May 9, 2012

Related U.S. Application Data
(60) Provisional application No. 61/187,708, filed on Jun. 17, 2009.

(51) Int. Cl. G01N 33/566

(52) U.S. Cl. 435/7.92; 436/501

(57) ABSTRACT

Methods for diagnosis of and distinguishing between, urosepsis, sepsis, and urinary tract infections (UTIs) are disclosed The diagnostic methods are based on determining the level of NGAL protein in a bodily fluid sample, such as urine sample.

This application claims the benefit of the filing date of U.S. Provisional Patent Application No. 61/187,708, filed Jun. 17, 2009, the contents of which are hereby incorporated by reference.

All patents, patent applications and publications cited herein are hereby incorporated by reference in their entirety.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Current tests for UTIs include the leukocyte esterase (LE) test, which detects the presence in the urine of an esterase enzyme released by white blood cells. The presence of leukocyte esterase is a sign of inflammation, which is most commonly caused by a urinary tract infection. Tests for nitrites in the urine (nitrituria) are also used in the diagnosis of UTI, with high nitrite levels indicating the presence of a urinary tract infection. Urine cultures may also be used, and require that samples of urine must be obtained by the “clean-catch” method or by inserting a sterile catheter through the urethra into the bladder. Diagnosis of bacteremia secondary to a UTI (i.e. urosepsis) typically requires both blood and urine cultures. Such methods are laborious and time-consuming.

### SUMMARY OF THE INVENTION

The invention provides methods for diagnosing urosepsis and UTI and methods for distinguishing urosepsis from localized UTI and from other forms of sepsis. The methods involve determining the amount of Neutrophil Gelatinase-Associated Lipocalin (NGAL) in a body fluid sample, for example urine.

The present invention is based, in part, on certain discoveries which are described more fully in the Examples section of the present application. For example, the present invention is based, in part, on the discovery that levels of NGAL protein in the urine of patients with urosepsis are higher than the levels of NGAL in the urine of patients with UTI, patients with other forms of sepsis, patients with acute kidney injury (AKI), and control patients. The invention is also based on the discovery that the level of NGAL protein in the urine is higher in patients with UTI as compared to patients with patients with sepsis (other than urosepsis), patients with AKI, and control patients. Thus, the present invention provides methods for diagnosis of UTI and urosepsis, and for distinguishing between urosepsis, sepsis, and UTI, and compositions and kits for use in such diagnostic methods.

In one embodiment, the present invention provides a method for determining whether a subject has urosepsis, the method comprising determining the concentration of NGAL protein in a urine sample from a subject, wherein a concentration of NGAL in the urine sample that exceeds a threshold amount indicates that the subject has urosepsis, and wherein a concentration of NGAL in the urine sample that is less than the threshold amount indicates that the subject does not have a urosepsis. In one embodiment, the threshold amount is between about 300 ng/ml and about 1300 ng/ml.

In another embodiment, the present invention provides a method for determining whether a subject has a UTI, the method comprising determining the concentration of NGAL protein in a urine sample from a subject, wherein a concentration of NGAL in the urine sample that exceeds a threshold amount indicates that the subject has a UTI, and wherein a concentration of NGAL in the urine sample that is less than the threshold amount indicates that the subject does not have a UTI. In one embodiment, the threshold is from about 150 ng/ml to about 500 ng/ml.

In another embodiment, the present invention provides a method for distinguishing whether a subject has urosepsis or some other form of sepsis, the method comprising determining the concentration of NGAL protein in a urine sample from a subject, wherein a concentration of NGAL in the urine sample that exceeds a threshold amount indicates that the subject has urosepsis as opposed to some other form of sepsis, and wherein a concentration of NGAL in the urine sample that is less than the threshold amount indicates that the subject does not have urosepsis but may have some other form of sepsis. In one such embodiment, the threshold is from about 300 ng/ml to about 1300 ng/ml.

In another embodiment, the present invention provides a method for distinguishing whether a subject has urosepsis or a localized UTI, the method comprising determining the concentration of NGAL protein in a urine sample from a subject, wherein a concentration of NGAL in the urine sample that exceeds a threshold amount indicates that the subject has urosepsis as opposed to a localized UTI, and wherein a concentration of NGAL in the urine sample that is less than the threshold amount indicates that the subject does not have urosepsis but may have a localized UTI. In one such embodiment, the threshold is from about 700 ng/ml to about 1300 ng/ml

In such diagnostic methods the step of determining the amount of NGAL in the urine can comprise performing an immunoassay, such as an ELISA, to detect NGAL protein. In some embodiments, the methods further comprise adjusting the subject's treatment regimen based on whether the concentration of NGAL in the urine sample exceeds or is less than the threshold amount.

In another embodiment, the present invention provides diagnostic kits for determining whether a subject has a UTI or urosepsis, and/or for distinguishing between UTI and urosepsis, and/or for distinguishing between urosepsis and sepsis, such kits comprising, for example: a device for detecting NGAL protein in the urine; a positive control containing NGAL protiein; and instructions indicating threshold levels of NGAL above which a diagnosis of UTI or urosepsis can be made. In one embodiment, the diagnostic kits contain instructions indicating that NGAL cut-off levels that can be used to make a diagnosis of UTI or urosepsis, or to distinguish between the two or between urosepsis and sepsis. In one embodiment, the device in the diagnostic kits comprises an anti-NGAL antibody. In one embodiment, the device in the diagnostic kits comprises an ELISA plate, a urine dipstick, or a test strip.

These and other embodiments of the invention are further described in the following sections of the application, including the Detailed Description, Examples, Claims, and Drawings.

### BRIEF DESCRIPTION OF THE FIGURES

FIG. 1: Two patients in the UTI group with uNGAL of 5000 or above and one patient in the urosepsis group with NGAL of 5500 not shown. Patients with urosepsis had significantly higher uNGAL (mean±SD) compared to all groups (1392±1627 ng/ml vs 560±985 ng/ml for UTI, 252±508 ng/ml for sepsis, 421±715 ng/ml for AKI and 59±137 ng/ml for controls, p<0.01 respectively). Patients with UTIs had significantly higher uNGAL levels compared to sepsis, AKI, and control patients (p<0.01 compared to sepsis and controls, p<0.05 compared to AKI). Patients with sepsis and AKI both had significantly elevated uNGAL compared to the controls (p<0.01). There were no significant differences between uNGAL in patients with sepsis or AKI

FIG. 2: There is a significant difference in the amount of NGAL in the patients with trace levels of leukocyte esterase (n=37, mean NGAL 143±217 ng/ml ) compared with patients with no leukocyte esterase (n=161, 53±124 ng/ml ). There is significantly more uNGAL in patients with 2+ leukocyte esterase (n=63, 928±1221 ng/ml ) compared to 1+ (n=43, 187±287 ng/ml ). There is no difference between patients with trace and 1+ leukocyte esterase.

FIG. 3: There is a weak correlation between serum white blood cells and uNGAL (r=0.23, p<0.01).

### DETAILED DESCRIPTION

The present invention is based, in part, on certain discoveries which are described more fully in the Examples section of the present application. For example, the present invention is based, in part, on the discovery that levels of NGAL protein in the urine of patients with bacteremia secondary to UTI (i.e. urosepsis) are higher than the levels of NGAL in the urine of patients with UTI, patients with other forms of sepsis, patients with AKI, and control patients, and the discovery that levels of NGAL protein in the urine of patients with UTI are higher than the levels of NGAL in the urine of patients with sepsis (not uroepsis), patients with AKI, and control patients. Thus, the present invention provides methods for diagnosis of UTI and urosepsis, and for distinguishing between UTI, urosepsis, and other forms of sepsis, and compositions and kits for use in such diagnostic methods.

### Abbreviations and Definitions

The abbreviation “NGAL” refers to Neutrophil Gelatinase Associated Lipocalin. NGAL is also referred to in the art as human neutrophil lipocalin, siderocalin, a-micropglobulin related protein, Scn-NGAL, lipocalin 2, 24p3, superinducible protein 24 (SIP24), uterocalin, and neu-related lipocalin. These alternative names for NGAL may be used interchangeably herein. Unless stated otherwise, the term “NGAL”, as used herein, includes any NGAL protein, fragment, or mutant that is expressed in the kidney, and which can be detected in a bodily fluid such as urine. In some embodiments the NGAL protein is wild-type human NGAL.

The abbreviation “uNGAL” is an abbreviation for urinary NGAL and refers to NGAL in the urine.

The abbreviation “UTI” refers to a urinary tract infection.

The abbreviation “AKI” refers to acute kidney injury.

The abbreviation “ROC” refers to receiver operating characteristic. ROC curves are widely used in the art for assessing diagnostic and prognostic tests. See, for example, Zweig & Campbell, (1993), “Receiver-operating characteristic (ROC) plots: a fundamental evaluation tool in clinical medicine”. Clinical chemistry 39 (8): 561-577; and Zou et al., (2007). “Receiver-operating characteristic analysis for evaluating diagnostic tests and predictive models.” Circulation, 6; 115(5): 654-7; and Lasko et al., (2005), “The use of receiver operating characteristic curves in biomedical informatics.” Journal of Biomedical Informatics, 38(5):404-415, the contents of each which are hereby incorporated by reference.

The abbreviation “AUC” refers to area under the curve, such as the area under an

ROC curve.

The term “urosepsis” is used herein in accordance with its normal meaning in clinical medicine, and refers to bacteremia that is secondary to a UTI

The term “sepsis” is used herein in accordance with its normal meaning in clinical medicine, and refers to bacteremia of any cause, which can include urosepsis. In contexts dealing with distinguishing between urosepsis and sepsis, the term sepsis may be used to refer to bacteremia with a cause other than UTI. Whether or not the term “sepsis” is intended to encompass urosepsis will be apparent from the context in which the term is used.

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

### DESCRIPTION

The invention provides that uNGAL levels are significantly higher in patients with bacteremia secondary to UTI (urosepsis) compared to patients with a UTI alone, patients with bacteremia from other sources (i.e. sepsis, but not urosepsis), and control patients, and also provides that uNGAL levels are significantly higher in patients with UTI compared to patients sepsis (but not urosepsis), and control patients.

Sepsis, including urosepsis, is a major cause of morbidity and mortality in hospitalized patients. Identification of the source of the infection can help tailor therapy and may improve patient outcomes. Current standard of care for the diagnosis of sepsis relies on the use of blood cultures, which can take days before results are given. A rapid test for the diagnosis of sepsis, including urosepsis, and for distinguishing between urosepsis and sepsis of other origins, will allow for earlier use of antibiotics and the use of more appropriate antibiotics, and will improve patient outcomes.

In one aspect of the invention, levels of NGAL protein in a bodily fluid, such as urine, that exceed a certain threshold amount can be used to diagnose urosepsis and to distinguish between urosepsis and sepsis of other origins (not secondary to UTI) and to distinguish between urosepsis and localized UTIs. It is a discovery of the invention that, in the study performed (see Example 1, control patients had mean uNGAL concentrations of 59 ng/ml, patients with urosepsis had mean uNGAL concentrations of 1392 ng/ml, patients with localized UTI had mean uNGAL concentrations of 568 ng/ml, patients with sepsis other than urosepsis had mean uNGAL concentrations of 252 ng/ml, and patients with AKI had mean uNGAL concentrations of 421 ng/ml.

Accordingly, in one embodiment the present invention provides methods for determining whether a subject has urosepsis, the methods comprising measuring the amount of NGAL protein in urine from the subject, wherein an amount of NGAL protein that exceeds a threshold level, such as a threshold level of about 300 ng/ml, or about 350 ng/ml, or about 400 ng/ml, or about 450 ng/ml, or about 500 ng/ml, or about 550 ng/ml, or about 600 ng/ml, or about 650 ng/ml, or about 700 ng/ml, or about 750 ng/ml, or about 800 ng/ml, or about 850 ng/ml, or about 900 ng/ml, or about 950 ng/ml, or about 1000 ng/ml, or about 1050 ng/ml, or about 1100 ng/ml, or about 1150 ng/ml, or about 1200 ng/ml, or about 1250 ng/ml, or about 1300 ng/ml, indicates that the subject has urosepsis. Conversely, an amount of NGAL protein that is less than such a threshold level can indicate that the subject does not have urosepsis.

In another embodiment the present invention provides methods for determining whether a subject has a UTI, the methods comprising measuring the amount of NGAL protein in urine from the subject, wherein an amount of NGAL protein that exceeds a threshold level, such as a threshold level of about 150 ng/ml, or about 200 ng/ml, or about 250 ng/ml, or about 300 ng/ml, or about 350 ng/ml, or about 400 ng/ml, or about 450 ng/ml, or about 500 ng/ml, indicates that the subject has a UTI. Conversely, an amount of NGAL protein that is less than such a threshold level can indicate that the subject does not have a UTI.

In another embodiment the present invention provides methods for distinguishing whether a subject has urosepsis or sepsis of some other origin (i.e. not secondary to a UTI), the methods comprising measuring the amount of NGAL protein in urine from the subject, wherein an amount of NGAL protein that exceeds a threshold level, such as a threshold level of about 300 ng/ml, or about 350 ng/ml, or about 400 ng/ml, or about 450 ng/ml, or about 500 ng/ml, or about 550 ng/ml, or about 600 ng/ml, or about 650 ng/ml, or about 700 ng/ml, or about 750 ng/ml, or about 800 ng/ml, or about 850 ng/ml, or about 900 ng/ml, or about 950 ng/ml, or about 1000 ng/ml, or about 1050 ng/ml, or about 1100 ng/ml, or about 1150 ng/ml, or about 1200 ng/ml, or about 1250 ng/ml, or about 1300 ng/ml, indicates that the subject has urosepsis. Conversely, an amount of NGAL protein that is less than such a threshold level can indicate that the subject does not have urosepsis but may have some other form of sepsis.

In another embodiment the present invention provides methods for distinguishing whether a subject has urosepsis or a localized UTI, the methods comprising measuring the amount of NGAL protein in urine from the subject, wherein an amount of NGAL protein that exceeds a threshold level, such as a threshold level of about 700 ng/ml, or about 750 ng/ml, or about 800 ng/ml, or about 850 ng/ml, or about 900 ng/ml, or about 950 ng/ml, or about 1000 ng/ml, or about 1050 ng/ml, or about 1100 ng/ml, or about 1150 ng/ml, or about 1200 ng/ml, or about 1250 ng/ml, or about 1300 ng/ml, indicates that the subject has urosepsis. Conversely, an amount of NGAL protein that is less than such a threshold level can indicate that the subject does not have urosepsis but may have a localized UTI.

In one embodiment the above methods can be used for the early detection of urosepsis, UTI, or sepsis, for example, before the onset of symptoms. Accordingly, in one aspect, the above methods be used to diagnose urosepsis, UTI, or sepsis in a subject who is not exhibiting signs of such a condition.

In another embodiment, the present invention provides a method for monitoring the progression of urosepsis, UTI, or sepsis in a subject, the method comprising measuring the amount of NGAL protein in a first bodily fluid sample taken from the subject and a second bodily fluid sample that is taken from the subject at a later period in time, wherein an amount of NGAL protein in the second sample that exceeds the amount of NGAL protein in the first sample, indicates that the urosepsis, UTI, or sepsis is worsening, and an amount of NGAL protein in the second sample that is less than the amount of NGAL protein in the first sample, indicates that the urosepsis, UTI, or sepsis is improving. In one embodiment, the first sample can be taken before the initiation of therapy for urosepsis, UTI, or sepsis, and the second sample can be taken after the initiation of such therapy. In another embodiment, both samples can be taken after the initiation of therapy. Thus, such methods can be used to monitor the effect of therapy on the progression of urosepsis, UTI, or sepsis in a subject.

In yet another embodiment, the present invention provides a solution to the problem of determining whether a subject is a candidate for treatment of urosepsis, UTI, or sepsis, the method comprising measuring the amount of NGAL protein in a bodily fluid, such as urine, from the subject, wherein an amount of NGAL protein that exceeds a threshold level indicates that the subject has is a candidate for treatment of urosepsis, UTI, or sepsis. Conversely an amount of NGAL protein that is less than the threshold level can indicate that the subject is not a candidate for treatment of urosepsis, UTI, or sepsis. In other embodiments, such methods also comprise subsequently treating the subject.

Another aspect of the invention provides a method of monitoring the effectiveness of a treatment for urosepsis, UTI, or sepsis in a subject, the method comprising the steps of: i) obtaining a baseline sample of a body fluid, such as urine, from the subject, ii) determining the level of NGAL in the baseline sample; iii) providing at least one treatment for the urosepsis, UTI, or sepsis, iv) obtaining at least one post-treatment sample of the body fluid from the subject; v) determining the level of NGAL in the post-treatment sample; and vi) evaluating the effectiveness of the treatment, based on comparing the level of NGAL in the post-treatment sample to the level of NGAL in the baseline sample.

It should be noted that in all of the embodiments above that deal with making an assessment relating to urosepsis, UTI, or sepsis based on detecting a level of NGAL in the urine that exceeds a threshold amount, ranges of uNGAL amounts can be used in the place of threshold values. For example, the threshold amounts provided above can be substituted with ranges.

For example, urosepsis may be indicated by an amount of uNGAL that falls within the range of about 300 ng/ml-2000 ng/ml, or about 350 ng/ml-2000 ng/ml, or about 400 ng/ml-2000 ng/ml, or about 450 ng/ml-2000 ng/ml, or about 500 ng/ml-2000 ng/ml, or about 550 ng/ml-2000 ng/ml, or about 600 ng/ml-2000 ng/ml, or about 650 ng/ml-2000 ng/ml, or about 700 ng/ml-2000 ng/ml, or about 750 ng/ml-2000 ng/ml, or about 800 ng/ml-2000 ng/ml, or about 850 ng/ml-2000 ng/ml, or about 900 ng/ml-2000 ng/ml. Also, the upper end of each of the preceding ranges can be adjusted, for example to about 2200 ng/ml, or about 2400 ng/ml, or about 2500 ng/ml, or about 2600 ng/ml, or about 2800 ng/ml, or about 3000 ng/ml, or more. uNGAL measurements falling below one of such ranges may indicate that the subject does not have urosepsis but may have a localized UTI or some other form of sepsis.

Similarly, UTI may be indicated by an amount of uNGAL that falls within the range of about 100 ng/ml-500 ng/ml, or about 150 ng/ml-500 ng/ml, or about 200 ng/ml-500 ng/ml, or about 250 ng/ml-500 ng/ml, or about 300 ng/ml-500 ng/ml, or about 350 ng/ml-500 ng/ml. Also, the upper end of each of the preceding ranges can be adjusted, for example to about 600 ng/ml, or about 700 ng/ml, or about 800 ng/ml, or about 900 ng/ml, or about 1000 ng/ml, or more. uNGAL measurements falling below one of such ranges may indicate that the subject does not have a UTI but may have sepsis (not urosepsis). Conversely, uNGAL measurements falling above one of such ranges may indicate that the subject has urosepsis.

The diagnostic methods described herein can be combined in various ways. Furthermore, the following description applies to all of the diagnostic methods described herein.

All of the diagnostic methods of the invention, such as those described above, can comprise one or more additional steps. The diagnostic methods of the invention may comprise one or more steps for obtaining the bodily fluid sample from the subject, for example using the methods described herein. The diagnostic methods of the invention may comprise one or more steps for treating the bodily fluid sample from the subject, for example using the methods described herein. The diagnostic methods of the invention may comprise one or more steps for detecting and/or measuring NGAL levels in the bodily fluid sample, for example using the methods described herein. The diagnostic methods of the invention may comprise one or more steps for treating the subject or altering the subject's treatment based on the level of NGAL detected and/or whether the measured NGAL level is greater or less than the chosen cut-off level or range. For example, if the subject's NGAL level suggests a diagnosis of urosepsis the subject may be treated for urosepsis, and if the subject's NGAL level suggests a diagnosis of sepsis of some other origin (i.e. not urosepsis) the subject may be treated for that sepsis, and if the subject's NGAL level suggests a diagnosis of UTI the subject may be treated for UTI.

According to the methods of the invention, such as the diagnostic methods described above, the bodily fluid can be any sample in which NGAL can be detected, including, but not limited to, blood, serum, or urine. In preferred embodiments the bodily fluid is urine.

Also according to the methods of the invention, the subject or patient can be any animal that is susceptible to UTI, urosepsis, or sepsis. In some embodiments the subjects are rodents, such as mice. In some embodiments, the subjects are cows, pigs, sheep, goats, cats, horses, dogs, and/or any other species of animal used as livestock or kept as pets. In preferred embodiments the subjects are human subjects. In some embodiments, the subjects are already suspected to have a UTI, sepsis, or urosepsis before testing according to the methods of the invention.

In certain embodiments, the NGAL protein detected and/or measured in the methods of the present invention has an amino acid sequence as defined by one of the following GenBank accession numbers, NP_{—}005555 (human NGAL), CAA67574 (human NGAL), P80188 (human NGAL), AAB26529 (human NGAL), P11672 (mouse NGAL), P30152 (rat NGAL), AAI132070 (mouse NGAL), AAI132072 (mouse NGAL), AAH33089 (human NGAL), and CAA58127 (human NGAL), or is a homolog, variant, derivative, fragment, or mutant thereof, and/or has at least 80% sequence identity, e.g., 85%, 90%, 95%, 98% or 99% sequence identity, with one of the above sequences.

In certain embodiments of the invention, it can be desirable to use a positive control for the detection of NGAL. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

As described herein, in certain embodiments, the present invention provides methods for determining whether a subject has UTI, urosepsis, or sepsis, and methods for distinguishing between such conditions, the methods comprising measuring the amount of NGAL protein in a bodily fluid, such as urine, from the subject, wherein an amount of NGAL protein that exceeds a threshold level or falls within a certain range indicates that the subject has a particular condition. In addition to the threshold amounts and ranges specified herein, a threshold level or range can also be selected by reviewing the data provided in the Examples section of this application, and selecting a threshold level that is sufficiently high that it is more likely than not that a subject having that level of NGAL will have the condition to be diagnosed (e.g. UTI, urosepsis, or sepsis), or will have a urosepsis as opposed to sepsis, or will have urosepsis as opposed to UTI, or vice versa. In addition, one of skill in the art can used standard statistical methods, such as ROC analysis, to test the diagnostic/prognostic value of certain threshold levels, and can then select the appropriate threshold level accordingly, based on standard methodologies known and used in the art.

It should also be noted that, although the amounts of NGAL described herein are generally referred to in terms of ng/ml NGAL, NGAL can also be measured and/or represented in other units, including, but not limited to measurements of the amount of NGAL relative to creatinine (e.g μg NGAL/g creatinine), or by any other units, and it should be understood that amounts of NGAL measured and/or represented in other units can be equivalent to the amounts and ranges described herein in terms of ng/ml NGAL. The present invention is not limited to methods that comprise measuring ng/ml NGAL. For example, an amount of NGAL that is represented herein as 100 ng/ml can also be represented in terms of, and encompasses, alternative measurements/units that correspond to the same amount of NGAL, e.g. the same amount of NGAL expressed in terms of mass alone (e.g. ng), or in terms of concentration expressed as μg/g creatinine, or in any other units. One of skill in the art can readily make the necessary conversions between units.

Furthermore, it should be noted that threshold levels or ranges of NGAL other than those specifically described herein may be used in accordance with the invention. It is a discovery of the invention that NGAL levels are higher in the urine of subjects with urosepsis as compared to subjects with other forms of sepsis, or with localized UTI, in control subjects. The mean levels of uNGAL in such groups (control, UTI, urosepsis, and sepsis groups) may vary in different groups of subjects or depending on the methodology used to measure NGAL levels. Accordingly, the present invention provides for the general concept of using uNGAL levels to diagnose urosepsis, UTI, and/or sepsis, and to distinguish urosepsis from other forms of sepsis and from localized UTI, and not only methods that rely on the specific thresholds and ranges provided herein.

In certain embodiments, other biomarkers can be assessed in addition to NGAL in order to determine whether a subject has UTI, urosepsis, or sepsis. For example, the present invention provides that, in addition to having a high level of urinary NGAL, UTI patients can also have one or more of: (i) high urine nitrites and/or (ii) a high leukocyte esterase (LE). Similarly, the present invention provides that, in addition to having a high level of urinary NGAL, urosepsis patients can also have positive urine and/or blood cultures. Likewise, the present invention provides that, in addition to having a high level of urinary NGAL, sepsis patients can also have positive blood cultures (but likely negative urine cultures). The diagnostic methods of the invention can be used in conjunction with these and other diagnostic methods known to be useful for the diagnosis of UTIs, urosepsis and sepsis.

According to the methods of the invention, samples of a bodily fluid can be obtained and/or tested using any means. For example, methods for collecting, handling and processing urine, blood, serum and plasma, and other body fluids, are well known in the art and can be used in the practice of the present invention. In some embodiments, two or more consecutive or subsequent samples of a body fluid can be taken. Depending upon the circumstances, including the level of NGAL in a sample and the clinical condition of the subject, the subject's body fluid can be sampled daily, or weekly, or within a few weeks, or monthly or within a few months, semi-annually, annually, or within several years, and at any interval in between. Repeat sampling can be done at a period of time after treatment to detect any change in disease status. Sampling need not be continuous, but can be intermittent (e.g., sporadic). In some embodiments, it is not necessary to obtain and keep a sample of the bodily fluid from the subject. For example, in some embodiments, the subject can urinate onto a test strip, for example a test strip of the type used in pregnancy testing kits. In other embodiments, a sample of bodily fluid, such as blood from a pin prick, can be applied onto a test strip—for example a test strip similar to those used for blood typing.

Although generally the sample of a bodily fluid, such as blood or urine, is obtained from a subject and tested by a laboratory or by a medical professional (for example using an automated urinalysis machine configured to test for NGAL, or an nNGAL testing kit, e.g. a urine dipstick based kit, or an ELISA based kit), home-testing kits are also within the scope of the present invention. In one aspect, the present invention comprises a kit for performing the methods of the invention, containing, for example, a device for detecting NGAL protein in the urine, and optionally including a positive control containing NGAL protien, and optionally including instructions, for example regarding the threshold levels of NGAL above which a diagnosis of UTI, urosepsis, or sepsis can be made. The device in such kits can comprise, for example, an ELISA plate, a dipstick or a test strip to be dipped in a urine sample or to have a sample or urine applied thereto, or a stick on which the subject should urinate. In some embodiments, such devices are configured such that they give a positive result only if the level of NGAL exceeds a threshold level, such as one of the threshold levels described herein. Methods for making and using such devices are well known in the art. Kits (ELISA kits), antibodies, and other reagents for detection of NGAL in the urine are commercially available, e.g. from Bioporto Diagnostics A/S and from R & D Systems, and can be used to make a kit according to the present invention. Such kits can be used by subjects themselves (e.g. home testing kits) or can be used by medical or laboratory staff

The present invention also provides methods based on measuring the levels of circulating NGAL, as opposed to urinary NGAL. Blood sampling is a routine clinical procedure, and blood samples of individuals may have been stored and preserved, providing a valuable database of historical samples that may be used to predict the progression disease in certain patients.

According to the methods of the invention, the presence and/or amount of NGAL protein in a bodily fluid, such as urine, can be detected and/or measured using any means known in the art. For example, in one embodiment, NGAL protein can be detected using antibodies that are specific to NGAL. Any antibody, such as a monoclonal or polyclonal antibody, that binds to NGAL can be used. For example, monoclonal antibodies that bind to NGAL are described in “Characterization of two ELISAs for NGAL, a newly described lipocalin in human neutrophils”, Lars Kjeldsen et al., (1996) Journal of Immunological Methods, Vol. 198, 155-16, the contents of which are herein incorporated by reference. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002). Further non-limiting examples of antibodies that can be used to detect NGAL protein in the methods of the invention are also provided in the Examples. Antibodies that bind to NGAL are also available commercially, for example from the Antibody Shop, Copenhagen, Denmark, as HYB-211-01, HYB-211-02, and NYB-211-05. In addition, one of skill in the art can readily produce antibodies that bind to NGAL, or can have them produced by an antibody production company.

Any method can be used to detect and or measure the levels of NGAL protein, including, but not limited to, immunohistochemistry-based methods, immuno-blotting based methods, immunoprecipitation-based methods, affinity-column based methods (including immunoaffinity column based methods), ELISA-based methods, other methods in which an NGAL antibody is immobilized on a solid substrate (such as beads), and the like. In some such methods the antibody to NGAL, or a secondary or tertiary antibody that binds directly or indirectly to the NGAL antibody, can be labeled with a detectable moiety, such as a fluorescent moiety, a radioactive moiety, or a moiety that is an enzyme substrate and can be used to generate a detectable moiety, such as horse radish peroxidase. Such methods are well known in the art and can be used to detect the presence and/or measure the amount of NGAL in a bodily fluid sample, such as urine, without undue experimentation.

In circumstances where the amount of NGAL is to be measured, positive controls containing known amounts of NGAL protein can be used, for example for calibration purposes. NGAL protein for use as a positive control can be obtained from any source or produced by any method known in the art. For example, NGAL protein can be recombinantly produced. Methods for the recombinant production of proteins are well known in the art. For example, a nucleotide sequence encoding NGAL can be included in an expression vector containing expression control sequences and expressed in, and purified from, any suitable cell type, such as bacterial cells or mammalian cells. For example, for use as a positive control in the methods of the invention, recombinant NGAL can be produced as described in Yang, et al. (2002) Mol Cell 10, 1045-1056; Goetz et al. (2002) Mol. Cell 10, 1033-1043; Goetz et al. (2000) Biochemistry 39, 1935-1941; and Mori, et al. (2005) J. Clin Invest. 115, 610-621, the contents of which are hereby incorporated by reference.

In other aspects of the invention, a diagnosis can be based upon, or can include, detecting the presence of NGAL protein or mRNA in tissues, such as in tissues of the urinary tract, as opposed to in a bodily fluid such as urine, for example by detecting a high level of NGAL protein or mRNA, or by detecting a specific localization of NGAL protein or mRNA. Such methods can be used alone, or can be used in conjunction with one or more other methods, such as the methods described herein for detection of NGAL in urine or other bodily fluids or standard diagnostic methods based on the examination of biopsy samples, etc. Methods for assessing the expression and/or localization of NGAL protein or mRNA in tissues of the urinary tract or in the kidney in situ are also provided by the invention, for example methods wherein, for example, labeled agents that bind to NGAL protein or mRNA are delivered to a subject and can be visualized in vivo, for example using imaging techniques such as CAT scan- based techniques and MRI-based techniques.

Detection of NGAL mRNA or protein can be determined using standard techniques and methodologies known to those of skill in the art, for example using samples obtained by biopsy. For example, NGAL mRNA can be detected by in situ hybridization using probes specific for NGAL, or by any other method known to be useful for detection of specific mRNAs, including, but not limited to, PCR-based techniques. The sequence of NGAL, including human NGAL, is known in the art. Similarly, sequences of probes and primers that can be used to detect NGAL are known in the art. In addition, NGAL protein can be detected using antibodies that are specific to NGAL, e.g. monoclonal or polyclonal antibodies can be used. In addition, detection methods that can be used, include, but are not limited to, immunohistochemistry-based methods and the like. Antibodies that are specific to NGAL and that could be used to detect NGAL in the kidneys are known in the art. Monoclonal antibodies for NGAL, are described, for example, in “Characterization of two ELISAs for NGAL, a newly described lipocalin in human neutrophils”, Lars Kjeldsen et al., (1996) Journal of Immunological Methods, Vol. 198, 155-16, herein incorporated by reference in its entirety. Non-limiting examples of antibodies that can be used to detect NGAL protein are provided in the Examples. Antibodies that bind to NGAL are also available commercially, for example from the Antibody Shop, Copenhagen, Denmark, as HYB-211-01, HYB-211-02, and NYB-211-05. Typically, HYB-211-01 and HYB-211-02 can be used with NGAL in both its reduced and unreduced forms. An example of a polyclonal antibody for NGAL is described in “An Iron Delivery Pathway Mediated by a Lipocalin”, Jun Yang et al., Molecular Cell, (2002), Vol. 10, 1045-1056, herein incorporated by reference in its entirety. To prepare this polyclonal antibody, rabbits were immunized with recombinant gel-filtered NGAL protein. Sera were incubated with GST-Sepharose 4B beads to remove contaminants, yielding the polyclonal antibodies in serum, as described by the applicants in Jun Yang et al., Molecular Cell (2002).

Those skilled in the art will recognize, or be able to ascertain using no more than routine experimentation, many equivalents to the specific embodiments of the invention described herein. Such equivalents are intended to be within the scope of the present invention.

The invention is further described by the following non-limiting Examples.

### EXAMPLE

Enrolled patients were over 18 years of age presenting to the Emergency Department (ED) at three different sites. Patients were excluded if they were admitted for less than 24 hours, in end stage renal disease, already receiving hemodialysis, or did not receive serial creatinine measurements while in the hospital. At study entry, demographic data including gender, age, medical history and previous diagnosis of kidney disease were recorded. Patients next provided up to ten milliliters of urine. Urine samples were spun in a centrifuge for 7 minutes, and stored at −80° C. At a later date, the amount of NGAL present in these urine samples was quantified by both Western Blot and ELISA assays. In addition, urine Na and urine urea, urine specific gravity, urine cells were measured by standard lab techniques. After patients provided urine, their subsequent hospital course was followed using electronic medical records. All patients enrolled in the study had their inpatient history followed from the date of admission to the date of discharge. The following information was collected during their inpatient admission: laboratory studies including electrolyte panels, urinalysis, and urine microscopy, medication history, procedure history, imaging history, and complication history.

722 consecutive patients presenting to the Emergency Department (ED) were enrolled. 9 patients had bacteremia secondary to UTI (urosepsis), defined as positive blood and urine cultures with the same pathogen, 65 had culture-positive UTIs, and 25 had culture-positive bactermia from other sources (sepsis). These patients were case matched on age, race, sex, and baseline serum creatinine to patients without any positive cultures. Additional patients without positive cultures were matched on presenting serum creatinine and included in the control group. Comparisons were also made to patients with AKI.

Patient characteristics are listed in Table 1:

| | | | | | Controls |
|---|---|---|---|---|---|
| | Urosepsis (n = 9) | UTI (n = 65) | Sepsis (n = 25) | AKI (n = 36) | (n = 196) |
| Age | 61 (17) | 72 (23) | 67 (20) | 67 (21) | 67 (21) |
| Female (%) | 67 | 70 | 50 | 61 | 58 |
| Hispanic (%) | 56 | 42 | 54 | 42 | 52 |
| Baseline Creatinine | 1.0 (0.3) | 1.0 (0.3) | 1.1 (0.5) | 1.3 (0.9) | 1.0 (0.4) |
| ER Creatinine | 1.6 (0.9) | 1.2 (0.6)† | 1.9 (1.7)* | 2.5 (2.7)** | 1.2 (0.8) |
| Baseline GFR | 71 (20) | 78 (28) | 76 (28) | 71 (32) | 78 (36) |
| ER GFR | 47 (18)* | 64 (28)† | 59 (36) | 47 (30)** | 72 (32) |
| Temperature | 100.2 (2.2) | 99.3 (1.9)* | 100.0 (2.4)** | 98.9 (1.2)*** | 98.5 (0.9) |
| Serum WBCs | 15 (7) | 13 (7)** | 14 (6)** | 18 (39) | 11 (17) |
| Urine WBCs | too many to | 21 to 30 | 6 to 10 | 6 to 10 | 3 to 5 |
| (median) | count | | | | |
| NGAL ng/mL | 1392 (1627)** | 568 (991)**$ | 252 (508)**$# | 421 (715)**$## | 59 (137) |

*p < 0.05 compared the controls,
**p < 0.01 compared to controls
†p < 0.01 compared to AKI
$p < 0.01 compared to Urosepsis
#p < 0.01 compared to UTI,
##p < 0.05 compared to UTI
***p > 0.05 compared to sepsis

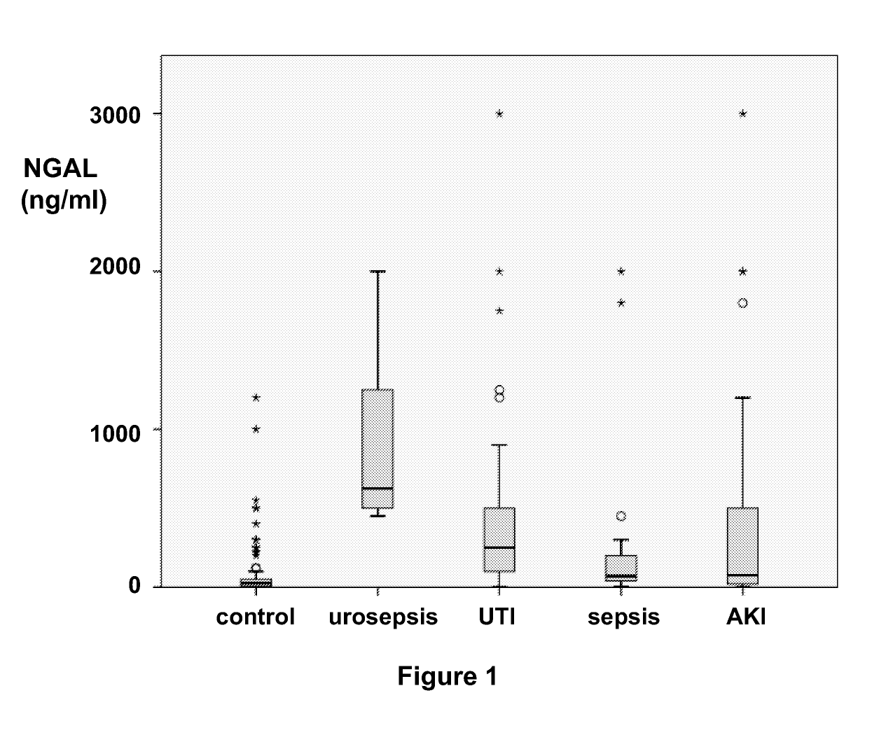

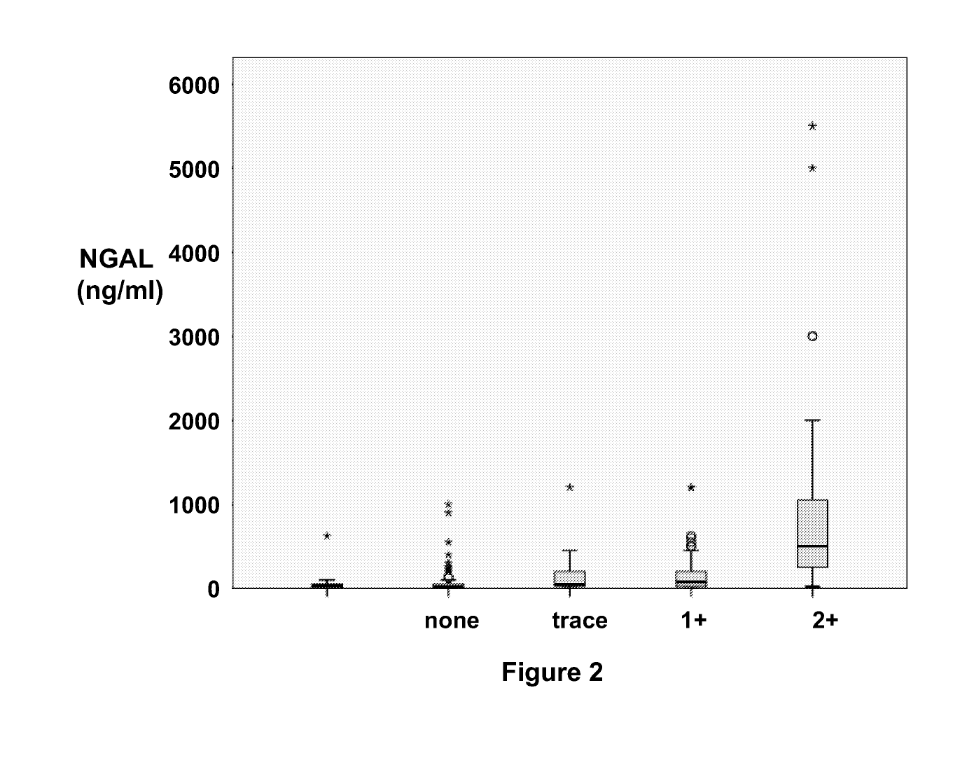

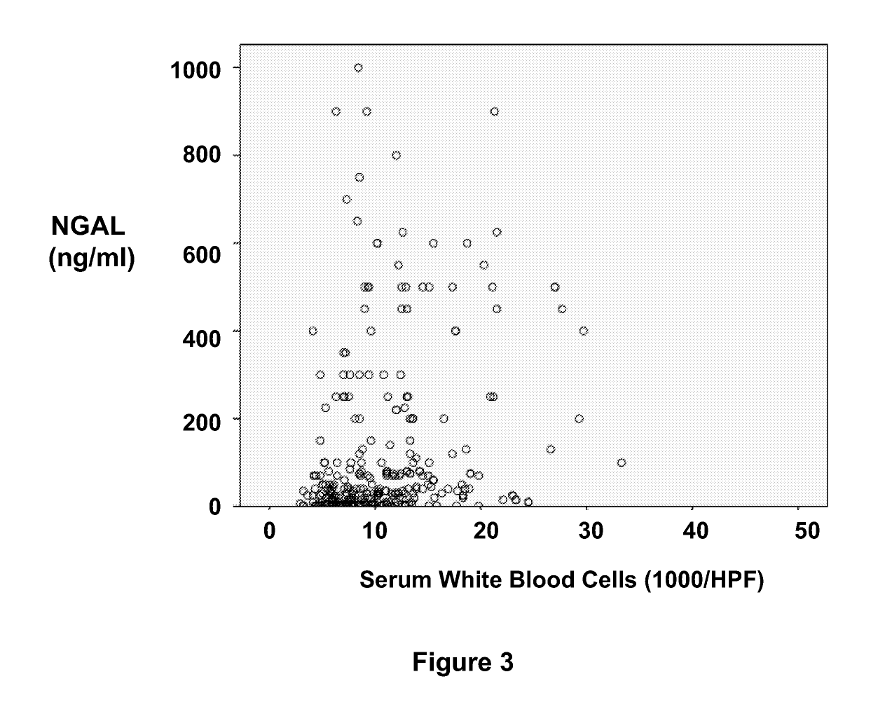

Patients with urosepsis had significantly higher uNGAL (mean±SD) compared to all groups (1392±1627 ng/ml vs 560±985 ng/ml for UTI, 252±508 ng/ml for sepsis, 421±715 ng/ml for AKI and 59±137 ng/ml for controls, p<0.01 respectively). Patients with UTIs had significantly higher uNGAL levels compared to sepsis, AKI, and control patients (p<0.01 compared to sepsis and controls, p<0.05 compared to AKI). Patients with sepsis and AKI both had significantly elevated uNGAL compared to the controls (p<0.01). There were no differences between uNGAL in patients with sepsis or AKI (FIG. 1). Receiver operating curve analysis showed uNGAL to have an AUC of 0.93 (0.90-0.97) for the diagnosis of urosepsis, compared to an AUC of 0.60 (0.51-0.70) for sepsis, and 0.81 (0.76-0.86) for UTI. A weak correlation existed between serum white blood cells and uNGAL (r=0.23, p<0.01, FIG. 3). Severity of urinalysis detected leukocyte esterase was associated with increased uNGAL levels. Compared to patients with no leukocyte esterase, uNGAL was significantly elevated in patients with trace leukocyte esterase (143±217 vs. 53±124 ng/ml, p<0.01), 2+ leukocyte esterase compared to 1+ (929±1221, 187±286, p<0.01). No difference existed between patients with trace and 1+ leukocyte esterase (FIG. 2).

### ***

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and rearranged in various ways within the scope and spirit of the invention.

What is claimed is:

1. A method for determining whether a subject has urosepsis, the method comprising determining the concentration of NGAL protein in a urine sample from a subject, wherein a concentration of NGAL in the urine sample that exceeds a threshold amount indicates that the subject has urosepsis, and wherein a concentration of NGAL in the urine sample that is less than the threshold amount indicates that the subject does not have urosepsis.

2. The method of claim 1, wherein a concentration of NGAL in the urine sample that is less than the threshold amount indicates that the subject does not have urosepsis but may have sepsis of some other origin or may have a localized urinary tract infection (UTI).

3. The method of claim 1, wherein the threshold amount is between about 300 ng/ml and about 1300 ng/ml.

4. The method of claim 1, wherein the threshold amount is between about 500 ng/ml and about 1300 ng/ml.

5. The method of claim 1, wherein the threshold amount is about 300 ng/ml.

6. The method of claim 1, wherein the threshold amount is about 400 ng/ml.

7. The method of claim 1, wherein the threshold amount is about 500 ng/ml.

8. The method of claim 1, wherein the threshold amount is about 600 ng/ml.

9. The method of claim 1, wherein the threshold amount is about 700 ng/ml.

10. The method of claim 1, wherein the determining step comprises performing an immunoassay to detect NGAL protein.

11. The method of claim 10, wherein the immunoassay is an ELISA.

12. The method of claim 1, further comprising adjusting the subject's treatment regimen based on whether the concentration of NGAL in the urine sample exceeds or is less than the threshold amount.

13. The method of claim 1, wherein the subject is a human.

14. A diagnostic kit for determining whether a subject has urosepsis, sepsis, or a localized UTI, the kit comprising: (a) a device for detecting NGAL protein in the urine; (b) a positive control containing NGAL protiein; and (c) instructions indicating a threshold level of NGAL above which a diagnosis of urosepsis, sepsis, or UTI can be made.

15. The diagnostic kit of claim 14, wherein the device for detecting NGAL protein in the urine comprises an anti-NGAL antibody.

16. The diagnostic kit of claim 14, wherein the device for detecting NGAL protein in the urine is an ELISA plate, a urine dipstick, or a test strip.
